(19) United States
(12) Patent Application Publication
(10) Pub. No.: US 2017/0121280 A1
(43) Pub. Date: May 4, 2017

(54) ORGANIC SOLVENT FOR PEROXIDES

(71) Applicant: ARKEMA FRANCE, Colombes (FR)

(72) Inventors: Jean-Luc DUBOIS, Millery (FR); Serge HUB, Villeurbanne (FR); Alfredo DEFRANCISCI, Félix Faure (FR)

(73) Assignee: ARKEMA FRANCE, Colombes (FR)

(21) Appl. No.: 15/317,707

(22) PCT Filed: Jun. 8, 2015
(86) PCT No.: PCT/FR2015/051506

§ 371 Date: Dec. 9, 2016

(30) Foreign Application Priority Data
Jun. 13, 2014 (FR) 1455384

(51) Int. Cl. C07C 407/00; C08F 4/36; C08G 63/78; C08K 5/14

(52) U.S. Cl. CPC C07C 407/006; C08K 5/14; C08F 4/36; C08G 63/78

(57) ABSTRACT

A peroxide solution of organic peroxide in an organic solvent, including at least one peroxide in a solvent, the solvent having a hydrophobic alcohol wherein the main carbonated chain having the alcohol function includes at least six carbon atoms, the solution also including a second, hydrophilic alcohol solvent representing a maximum of 15% wt. % of the solution. Also, a thermohardenable composition including said solution, and a method and a use relating to the composition.

### FIELD OF THE INVENTION

The invention is relative to an organic solvent and at least one peroxide, and also the more particular use of this solvent for a particular class of dangerous organic peroxides.

It is clearly understood that the application of this specific solvent to a dangerous peroxide, as a result of its decomposition and of its risk of exothermic decomposition, is particularly indicated, considering the advantage in the individual case, but that this is under no circumstances limited to such a use and can be entirely envisaged for other peroxides, in view of its properties.

Some peroxides are particularly dangerous and can, both during their manufacture and their transportation, very rapidly and exothermically decompose and can consequently result in a fire, indeed even an explosion. To avoid such risks, it is necessary to use a solvent of A type, defined as an organic solvent having a boiling point of greater than 160° C., for their manufacture and their transportation.

A well known class of such peroxides are ketone peroxides.

### STATE OF THE ART

It will be possible the documents DE 3512829 or FR 2445341 as basis for the disclosure of a state of the art in the field.

The document U.S. Pat. No. 3,557,009 is also known, which document discloses a few examples of peroxide solutions comprising an alcohol as organic solvent, in the case in point 2-ethylhexanol, without, furthermore, using a component of the family of phthalates. It has been found by the applicant company that, when a single alcohol is used as organic solvent for a peroxide, the composition is not stable over time.

Two tests were carried out and show, for one, that the synthesis with a single hydrophobic alcohol, in the case in point 2-ethylhexanol, and also the synthesis with a single hydrophilic alcohol, in the case in point DAA, does not make it possible to obtain a stable solution, at least after a certain period of time.

Test 1:

Synthesis of an MEKP mixture using only the hydrophobic alcohol 2-ethylhexanol.

The peroxide obtained has a cloudy/milky appearance. We were able to separate, by a stage of settling at 16° C., a certain amount of aqueous phase, which was discarded. We recovered the remaining organic phase, which still had this cloudy appearance, and we neutralized it with 10% of a 3% solution of TBA (tributylamine) in 2-ethylhexanol. The product obtained still exhibited this cloudy and milky appearance. After a few hours (2 hours) spent in a refrigerator at 0° C., an aqueous phase separates: it is possible to see several drops of a liquid deposited at the bottom of the flask containing the sample. The product is thus not physically stable (separation of phases) under realistic storage conditions for several countries in winter.

Test 2:

Synthesis of an MEKP mixture using only the hydrophilic alcohol DAA (diacetone alcohol).

The peroxide obtained exhibits a single phase which is colorless and transparent; it is thus impossible for us to remove the aqueous phase, rich in residual sulfuric acid, by a stage of separation by settling.

The neutralization stage is carried out by adding 10% of a 3% solution of tributylamine in DAA. The product obtained exhibits just one phase. This sample could not pass or satisfy an accelerated aging test of 7 (seven) days at 50° C. as it lost more than 10% of the starting active oxygen content. The product is thus not thermally stable and it probably cannot have a storage life at ambient temperature of greater than 6 (six) months at ambient temperature.

The documents GB 1 072 728, WO 2011/033232 and U.S. Pat. No. 5,808,110 are also known, which documents mention the use of an alcohol as organic solvent for a peroxide composition. Nevertheless, the alcohol is always used in combination with another nonalcoholic solvent, which results in certain negative characteristics and in particular does not make it possible, especially for some types of peroxides, to guarantee an optimum stability over time of the peroxide composition.

Currently, dimethyl phthalate or diisobutyl phthalate is conventionally used as solvent for peroxides of this type. The major disadvantage of these solvents lies in the fact that they belong to the family of phthalates, which are chemicals, some of which are regarded as toxic and exhibiting environmental disadvantages.

Methyl benzoate and aliphatic esters (in particular TXIB®) are also known as main solvent for such peroxides but none of these solvents is completely satisfactory.

This is because it is desirable, in some applications, to have a solution of solvent and at least one peroxide which exhibits a viscosity which is as low as possible in order for it to be easily dispersed as fine droplets, for example via a nozzle or a gun.

In addition, an important criterion is the absence of release of gas from the mixture (peroxide+solvent) over time, for example during storage, this gas marking a significant deterioration in the properties/characteristics of the mixture.

Finally, when a peroxide requiring the presence of a solvent is prepared, a solution with an aqueous phase and an oily or organic phase (mixture of said organic solvent with the peroxide or peroxides) containing the peroxide or peroxides is conventionally present. Subsequently, only the oily phase is of use and thus recovered, so that it is necessary for the settling, or the separation, of the two organic and aqueous phases to be carried out as best as possible. The criterion of separation by settling of the solvent composition is thus important.

Finally, there exists on the market no solvent which makes it possible to ensure optimum safety for peroxides of the ketone type and which is capable of also fulfilling as best as possible the combined properties and characteristics indicated above.

### BRIEF DESCRIPTION OF THE INVENTION

It has been found, after various experiments and contrary to the teachings of the state of the art, that a specific alcohol mixture, namely a hydrophobic alcohol and a hydrophilic alcohol, makes it possible to meet the requirements targeted above and in particular to offer an optimum stability of the peroxide composition.

Furthermore, the organic peroxide solution according to the invention does not have any phthalate, ester or generally any of the organic solvents conventionally used to date to dissolve organic peroxides and in particular certain families of organic peroxides. This is because the solvents used to date, in particular conventional phthalates or esters, used alone or as a mixture, all exhibit particular disadvantages.

The present invention intends to solve all of the technical problems related to these organic solvents by a combination of at least two alcohols, which alone makes it possible to store, stable, the solution of peroxides, while overcoming the disadvantages of solutions of the prior art.

A person skilled in the art knows that alcohols are polar components liable to exhibit interaction with water. He will thus not be inclined to envisage their use as solvent for peroxide in a solution furthermore containing an aqueous phase, the separation of the two phases being rendered difficult by the use of an alcohol.

Thus, the present invention relates to a solution of organic peroxide in an organic solvent, comprising at least one peroxide present in a solvent, the solvent consisting of a hydrophobic alcohol, the main carbon chain of which, carrying the alcohol functional group, comprises at least six carbon atoms, characterized in that it additionally comprises a second hydrophilic alcohol solvent present at most at a level of 15% by weight of the solution.

According to a preferred embodiment of the invention, the peroxide is chosen from ketone peroxides. This is because the invention exhibits a major advantage for this family of peroxides.

The present invention exhibits a consequent additional advantage in that an alcohol (or more specifically an alcohol mixture) used as solvent and corresponding to the requirements targeted above can preferably consist of a renewable product, that is to say a product obtained from renewable starting materials, preferably vegetable materials, the stock of which can consequently be built up again over a short period on the human scale.

Practically, the renewable nature of a component originates from the fact that at least a portion of the carbon atoms is of renewable origin, it being possible for this portion of renewable origin to be determined according to the standard ASTM D 6866-06.

Unlike the materials resulting from fossil materials, renewable starting materials contain ¹⁴C. All the samples of carbon drawn from living organisms (animals or plants) are in fact a mixture of 3 isotopes: ¹²C (representing approximately 98.892%), ¹³C (approximately 1.108%) and ¹⁴C (traces: 1.2×10⁻¹⁰%). The ¹⁴C/¹²C ratio of living tissues is identical to that of the atmosphere. In the environment, ¹⁴C exists in two predominant forms: in the form of carbon dioxide gas (CO₂) and in the organic form, that is to say in the form of carbon incorporated in organic molecules.

In a living organism, the ¹⁴C/¹²C ratio is kept constant by the metabolism as the carbon is continually exchanged with the external environment. As the proportion of ¹⁴C is constant in the atmosphere, it is the same in the organism, as long as it is living, since it absorbs this ¹⁴C in the same way as ambient ¹²C. The mean ¹⁴C/¹²C ratio is equal to 1.2×10⁻¹².

¹²C is stable, that is to say that the number of ¹²C atoms in a given sample is constant over time. ¹⁴C is radioactive; the number of ¹⁴C atoms in a sample decreases over time (t), its half life being equal to 5730 years. The ¹⁴C content is substantially constant from the extraction of renewable starting materials.

Preferably, the hydrophobic alcohol of the organic solvent according to the invention thus consists of octan-2-ol, more preferably still obtained directly from the castor oil plant.

According to one possibility offered by the invention, the hydrophobic alcohol of the organic solvent according to the invention consists of 2-ethylhexanol.

Other characteristics and distinguishing features of the primary mixture of the invention are presented below:
preferably, the proportion of solvent to the proportion of peroxide becomes established between 10% and 90% by weight of the solution, preferably between 35% and 65%;
according to a distinguishing feature of the invention, the peroxide is methyl ethyl ketone peroxide;
preferably, the second hydrophilic alcohol consists of diacetone alcohol;
according to a possibility offered by the invention, the solution consists solely of the organic solvent, a mixture of a hydrophobic alcohol and of a hydrophilic alcohol, and the organic peroxide.

The invention also relates to a polymerizable and/or crosslinkable thermosetting composition comprising at least monomer units and/or a polymer, characterized in that it comprises a solution as defined above.

Thermosetting resins acceptable for being crosslinked in accordance with the process according to the present invention comprise unsaturated polyester resins, vinyl ester resins, (meth)acrylate resins, polyurethanes, epoxy resins and the mixtures of these, such as the mixtures of unsaturated polyester resins and epoxy resins or mixtures of different unsaturated polyester resins.

Preferred resins are (meth)acrylate resins, unsaturated polyester resins and vinyl ester resins.

In the context of the present invention, the term “unsaturated polyester resins” and unsaturated polyester resin refer to the mixture of unsaturated polyester resin and of ethylenically unsaturated monomer component. The term “(meth)acrylate resin” refers to the mixture of acrylate or methacrylate resin and of ethylenically unsaturated monomer component. The unsaturated polyester resins and the acrylate resins defined above are well known to a person skilled in the art and are commercially available.

Unsaturated polyester resins suitable for being crosslinked according to the process of the present invention are known as ortho resins, iso resins, iso-NPG resins and dicyclopentadiene (DCPD) resins. Examples of such resins are resins of the maleic, fumaric, allylic, vinyl and epoxy type, bisphenol A resins, terephthalic resins and hybrid resins.

Vinyl ester resins comprise acrylic resins, based on, for example, methacrylate, diacrylate, dimethacrylate and oligomers thereof.

An unsaturated polyester or vinyl ester resin can contain a monomer. Examples of appropriate monomers are ethylenically unsaturated monomer components, such as styrene and styrene derivatives, such as o-methylstyrene, vinyltoluene, indene, divinylbenzene, vinylpyrrolidone, vinylsiloxane, vinylcaprolactam or stilbene but also diallyl phthalate, dibenzylideneacetone, allylbenzene, methyl methacrylate, methacrylate, (meth)acrylic acid, diacrylates, dimethacrylates, acrylamides, vinyl acetate, triallyl cyanurate, triallyl isocyanurate, allyl components which are, for example, used in optical applications (such as (di)ethylene glycol bis(allyl carbonate)), chiorostyrene, tert-butylstyrene, tert-butyl acrylate, butanediol dimethacrylate and the mixtures thereof.

Appropriate examples of reactive (meth)acrylate diluents are PEG 200 di(meth)acrylate, 1,4-butanediol di(meth)acrylate, 1,3-butanediol di(meth)acrylate, 2,3-butanediol di(meth)acrylate, triethylene glycol di(meth)acrylate, glycerol di(meth)acrylate, trimethylolpropane di(meth)acrylate, neopentyl glycol di(meth)-acrylate, dipropylene glycol di(meth)acrylate, tripropylene glycol di(meth)acrylate, PPG 250 di(meth)acrylate, tricyclodecane dimethylol di(meth)acrylate, 1,10-decanediol di(meth)acrylate, tetraethylene glycol di(meth)acrylate, trimethylolpropane tri(meth)acrylate, pentaerythritol tetra(meth)acrylate, glycidyl (meth)acrylate, (bis)maleimides, (bis)citraconimides, (bis)itaconimides and the mixtures of these.

The amount of ethylenically unsaturated monomer in the resin preferably represents at least 0.1% by weight of the unsaturated polyester or vinyl ester resin, more preferably at least 1% and more preferably still at least 5% by weight. The amount of ethylenically unsaturated monomer preferably represents at most equal to 50% by weight of the resin, more preferably at most 40% and more preferably still at most 35%.

The invention also relates to the use of two alcohols, including a hydrophobic alcohol exhibiting a main chain, carrying the alcohol functional group, comprising at least six carbon atoms, preferably octan-2-ol or 2-ethylhexanol, and a second hydrophilic alcohol present at most at a level of 15% by weight of the solution, as organic solvent of at least one peroxide, preferably chosen from ketone peroxides and more particularly consisting of methyl ethyl ketone peroxide, in order to form a solution as defined above.

The description which will follow is given solely by way of illustration and without limitation.

### DETAILED DESCRIPTION OF THE INVENTION

The present invention is intended first to ensure that the preparation of an organic peroxide of ketone type is made secure. Nevertheless, the invention provides a solution comprising an organic solvent and a peroxide which intrinsically comprises properties far superior to those of the prior art.

As regards the peroxide in the solution, it will preferably be chosen from the family of the ketone peroxides. The peroxides of this family all exist in the following form:

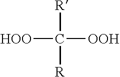

Mention may in particular be made, as examples of components belonging to the family of the ketone peroxides, of methyl ethyl ketone peroxide, acetal ketone peroxide, methyl isobutyl ketone peroxide, methyl isopropyl ketone peroxide and cyclohexanone peroxide.

The invention is certainly not limited to the use of the organic solvent with ketone peroxides and can be envisaged for all the other peroxides. The invention in the continuation is presented with tests relating solely to a ketone peroxide, more specifically methyl ethyl ketone peroxide, but additional tests have made it possible to show that highly satisfactory and similar results are obtained with different peroxides.

Mention will thus be made, by way of illustration of other peroxides which can be envisaged in the context of the present invention, of dialkyl peroxides (di(t-butyl) peroxide, dicumyl peroxide, for example), diacyl peroxides (dibenzoyl peroxide, dilauroyl peroxide, di(2,4-dichlorobenzoyl) peroxide, for example), hydroperoxides (t-butyl hydroperoxide, α-cumyl hydroperoxide, 1-phenylethyl hydroperoxide, for example), peroxyacids (peroxyacetic acid, p-nitroperoxybenzoic acid, for example), peroxyesters (t-butyl peroxyacetate, t-butyl peroxypivalate, t-butyl peroxybenzoate, for example), peroxyketals (1,1-di(t-butylperoxy)-3,3,5-trimethylcyclohexane, 1,1-di(t-butylperoxy)cyclohexane, 2,2-di(cumylperoxy)propane, for example), peroxydicarbonates (diisopropyl peroxydicarbonate, di(sec-butyl) peroxydicarbonate, di(2-butoxyethyl) peroxydicarbonate, for example), sulfonyl peroxides (acetyl cyclohexane sulfonyl peroxide, for example) and silyl peroxides (vinyltri(t-butylperoxy)silane, cumylperoxytrimethylsilane, for example).

As regards the alcohol in the solution, it has been found by the applicant company that the noteworthy properties presented above are obtained with alcohols exhibiting a main chain, that carrying the alcohol (OH) functional group, having at least six carbon atoms. This is because there has been found, with a pentanol, a notable deterioration in the results obtained with a hexanol.

In the continuation, the tests relating to the solution according to the invention are carried out with octan-2-ol and 2-ethylhexanol but it has been demonstrated that other alcohols corresponding to the main definition of the present invention exhibit comparable advantageous results.

As regards the polymerizable and/or crosslinkable composition according to the invention, it adds polymerizable monomer units and/or at least one crosslinkable polymer to the solution. In other words, the “methyl ethyl ketone peroxide+octan-2-ol/2-ethylhexanol” solution is more conventionally intended for the polymerization/crosslinking of thermosetting resins.

To this composition, different components or additives can optionally be added which can comprise metal salts intended to promote the decomposition of the organic peroxide to give free radicals, copromoters of the decomposition of the organic peroxide, such as tertiary aromatic amines, acetylacetone, ethyl acetoacetate or N,N-diethylacetoacetamide; UV protection agents; processing aids having the role of improving the final appearance during its use, such as fatty amides, stearic acid and its salts, ethylenebisstearamide or fluoropolymers; antifogging agents; antiblocking agents, such as silica or talc; fillers, such as calcium carbonate and nanofillers, such as, for example, clays; coupling agents, such as silanes; antistatic agents; nucleating agents; pigments; dyes; plasticizers; viscosity reducers and flame-retardant additives, such as aluminum or magnesium hydroxides.

This composition can be used to produce composite materials, such as resin/glass fiber or resin/carbon fiber or resin/plant fiber or resin/plastic fiber laminates.

It can also be used as binder in the production of reconstituted minerals, such as reconstituted marble, reconstituted granites or aluminum hydroxide (Al(OH)₃), to mention the commonest.

According to the invention, one or more crosslinking retarders can also be introduced into the composition, such as the antioxidant compounds of the family of hydroquinones and phenolic antioxidants.

These additives are generally used in contents of between 10 ppm and 10 000 ppm by weight, with respect to the final weight of resin. The plasticizers, the viscosity reducers and the flame-retardant additives can reach amounts much greater than 10 000 ppm.

Example of the Preparation of a Solution According to the Invention:

The solution according to the invention, namely comprising the organic solvent according to the invention and at least one peroxide, is prepared in a way which is completely conventional and well known to a person skilled in the art.

Furthermore, when the solvent according to the invention is used for the preparation/manufacture of the organic peroxide, such as, for example, in the case of ketone peroxides and more particularly of methyl ethyl ketone peroxide, said preparation or said manufacture is not in any way modified and only the peroxide used in the prior art as solvent is replaced by a solvent according to the present invention.

Tests Carried Out:

In the continuation, the solvents according to the invention which are tested are a mixture of octan-2-ol or 2-ethylhexanol and of diacetone alcohol and this solvent mixture is compared with the solvents typically used with organic ketone peroxides, namely TXIB (2,2,4-trimethyl-1,3-pentanediol diisobutyrate of the family of the aliphatic esters), DMP (dimethyl phthalate) and DIBP (diisobutyl phthalate), and also with an organic solution in which only a hydrophobic alcohol is used, namely 2-ethylhexanol.

All these solutions comprise exactly the same peroxide, in the case in point methyl ethyl ketone peroxide, and the same ratio of amount of the latter with respect to the organic solvent, this being the case in order to strictly compare the properties of the organic solvent and its interaction and its synergies with peroxide, the reader being reminded that additional tests have shown that the conclusions drawn from the experiments presented below are obtained in a substantially equivalent manner with other peroxides, in other words peroxides belonging to other families than that of the ketone peroxides.

Viscosity Measurement

The viscosity measurement is carried out on the solutions using a Haake Viscotester VT550 equipped with an NT spindle at 20° C. It is necessary to obtain a solution having a viscosity of less than 15 mPa·s in order to allow its use under special circumstances, in particular for its projection as fine droplets using a narrow nozzle or using a gun.

A viscosity of 13 mPa·s is recorded for the two solutions comprising methyl ethyl ketone peroxide and respectively octan-2-ol and also diacetone alcohol and 2-ethyl-hexanol with diacetone alcohol.

The solution comprising methyl ethyl ketone peroxide and DIBP exhibits a viscosity of 31 mPa·s, while those with TXIB and DMP both exhibit a viscosity value equal to 16 mPa·s.

Measurement of Gas Production

This measurement corresponds to the gassing test, which consists in measuring the deformation of a hermetically sealed low-density polyethylene (LDPE) bottle containing organic peroxide during a residence time of five days in an oven at 50° C. This test uses a 60 ml oval-shaped PE flask on which has been drawn a mark at 2.5 inches (i.e. 6.35 cm; an inch being equal to 2.54 centimeters) from the bottom of the plastic bottle. 30 ml of peroxide to be analyzed are deposited in this flask. The gassing test makes it possible to determine a factor F. This factor is calculated with regard to the difference in level between the mark drawn beforehand at the beginning of the test at 2.5 inches and the final level of peroxide measured after five days at 50° C.

An acceptable gassing test factor should be less than or equal to 2.

The detailed procedure is as follows:
an oval PE flask on which a mark at 2.5 inches has been drawn beforehand is tared with its stopper,
30 ml of peroxide to be analyzed are introduced into this flask,
this flask is weighed again and the weight of peroxide introduced is deduced therefrom,
the sides of the PE bottle are pressed until the level of the contents reaches a level of 2.5 inches and then the bottle is hermetically stoppered,
the flasks are left at ambient temperature for 48 h in order to confirm that each of the stoppers is satisfactorily airtight (the level of peroxide should still be at the level of the drawn line),
the flasks are deposited in a ventilated oven at 50° C. for 5 days,
at the end of the test, the flask is weighed and the loss in weight is deduced therefrom,
the difference in the level between the mark of 2.5 inches and the final level of the peroxide is measured. The gassing test factor (F) is calculated by assigning one point per each ⅛th of an inch (0.3175 cm) between the final level of liquid and the mark drawn initially at 2.5 inches. A factor F of less than or equal to 2 indicates that the peroxide passes the gassing test.

It is found, after a period of time of 5 days, that the interior volume of the bottle has remained stable (no apparent expansion of the bottle nor any excess pressure on opening said bottle) for the solution comprising the mixture based on octan-2-ol or 2-ethylhexanol and diacetone alcohol, whereas the excess pressure for the other solutions has caused a major deformation of the bottle due to the expansion of the gases given off by the emulsions under consideration.

More specifically, the solution comprising TXIB gave a factor F of 0.63, corresponding to a fall in level in the LDPE bottle of 0.2 cm.

Measurement of Separation by Settling of the Two Organic and Aqueous Phases

In this test, approximately 300 g of methyl ethyl ketone peroxide are synthesized by adding methyl ethyl ketone to each of the solvents, the two (octan-2-ol and 2-ethyl-hexanol) according to the invention and the three (TXIB, DMP and DIBP) according to the prior art, and by then adding a solution of acid and of aqueous hydrogen peroxide solution. From the end of the reaction between the hydrogen peroxide and MEK, each of these solutions exists as an emulsion having a cloudy appearance. They are placed in a separating funnel. The two phases are subsequently left to separate by settling.

It is found that the two phases, organic and aqueous, have completely separated after one minute in the two solutions according to the invention (octan-2-ol and 2-ethylhexanol), whereas, for the solutions according to the prior art, the times for separation of the two phases are approximately as follows:
for the solution with TXIB: between 10 and 15 minutes,
for the solution with DIBP: 10 minutes.

1. Solution of organic peroxide in an organic solvent, comprising at least one peroxide present in a solvent, the solvent consisting of a hydrophobic alcohol, the main carbon chain of which, carrying the alcohol functional group, comprises at least six carbon atoms, wherein the solution additionally comprises a second hydrophilic alcohol solvent present at most at a level of 15% by weight of the solution.

2. Solution as claimed in claim 1, wherein the peroxide is chosen from ketone peroxides.

3. Solution as claimed in claim 1, wherein the hydrophobic alcohol consists of octan-2-ol.

4. Solution as claimed in claim 1, wherein the hydrophobic alcohol consists of 2-ethylhexanol.

5. Solution as claimed in claim 1, wherein the proportion of solvent to the proportion of peroxide becomes established between 10% and 90% by weight of the solution.

6. Solution as claimed in claim 1, wherein the peroxide is methyl ethyl ketone peroxide.

7. Solution as claimed in claim 1, wherein the second hydrophilic alcohol consists of diacetone alcohol.

8. Solution as claimed in claim 1, wherein the solution consists of the organic solvent, a mixture of a hydrophobic alcohol and of a hydrophilic alcohol, and the organic peroxide.

9. Polymerizable and/or crosslinkable thermosetting composition comprising at least monomer units and/or a polymer, wherein the composition comprises a solution as claimed in claim 1.

10. A method of forming a solution, the method comprising using two alcohols, including a hydrophobic alcohol exhibiting a main chain, carrying the alcohol functional group, comprising at least six carbon atoms, and a second hydrophilic alcohol present at most at a level of 15% by weight of the solution, as organic solvent of at least one peroxide, in order to form a solution as claimed in claim 1.
